(19) United States
(12) Patent Application Publication
(10) Pub. No.: US 2025/0347097 A1
(43) Pub. Date: Nov. 13, 2025

(54) FOOD WASTE PROCESSING DEVICE AND CONTROL METHOD THEREOF

(71) Applicant: SAMSUNG ELECTRONICS CO., LTD., Suwon-si (KR)

(72) Inventors: Daegeon KIM, Suwon-si (KR); Hyeongyu IM, Suwon-si (KR); Namsoo PARK, Suwon-si (KR); Wonyoung SEO, Suwon-si (KR)

(73) Assignee: SAMSUNG ELECTRONICS CO., LTD., Suwon-si (KR)

(21) Appl. No.: 19/276,918

(22) Filed: Jul. 22, 2025

Related U.S. Application Data
(63) Continuation of application No. PCT/KR2024/002063, filed on Feb. 14, 2024.

(30) Foreign Application Priority Data
Mar. 30, 2023 (KR) 10-2023-0041747

(51) Int. Cl. E03C 1/266

(52) U.S. Cl. CPC E03C 1/2665

(57) ABSTRACT

A food waste disposal device includes: a treatment chamber including an inlet, a drain part connected to a sink drainpipe, and a byproduct outlet through which byproducts are discharged; a pulverizing transfer part rotatably disposed within the treatment chamber, and configured to grind and transfer an object introduced through the inlet; a storage part configured to store the byproducts discharged through the byproduct outlet; a drain valve configured to adjust an opening degree of the drain part; and a controller configured to control operations of the pulverizing transfer part and the drain valve to perform: in a first mode, an operation of opening the drain valve and rotating the pulverizing transfer part in a first direction to grind and transfer the object introduced through the inlet; and in a second mode, an internal cleaning operation of operating the pulverizing transfer part.

### CROSS-REFERENCE TO RELATED APPLICATIONS

This application is a continuation of International Application No. PCT/KR2024/002063, filed on Feb. 14, 2024, in the Korean Intellectual Property Receiving Office, which is based on and claims priority to Korean Patent Application No. 10-2023-0041747, filed on Mar. 30, 2023, in the Korean Intellectual Property Office, the disclosures of which are incorporated by reference herein in their entireties.

### BACKGROUND

### 1. Field

The disclosure relates to a food waste processing device and a method for controlling the same.

### 2. Description of Related Art

Typically, food waste disposal devices designed to grind and dispose of food waste may be installed in a sink or configured as standalone units, enclosed in a casing, allowing users to position them as desired.

The food waste disposal device installed in the sink may pulverize, compress, and dry the food waste introduced via the sink bowl, then transfer it to the storage part. The food waste disposal device, as previously described, grinds food waste using a pulverizing transfer part. Odors may be generated if food debris remains attached to the inner wall of the pulverizing transfer part without being transferred. In order to prevent such odor, the inside of the pulverizing transfer part should be cleaned periodically.

### SUMMARY

Provided are a device and a control method that may automatically clean the interior to remove any remaining food waste.

Additional aspects will be set forth in part in the description which follows and, in part, will be apparent from the description, or may be learned by practice of the presented embodiments.

According to an aspect of the disclosure, a food waste disposal device may include: a treatment chamber including an inlet, a drain part connected to a sink drainpipe, and a byproduct outlet through which byproducts are discharged; a pulverizing transfer part rotatably disposed within the treatment chamber, and configured to grind and transfer an object introduced through the inlet; a storage part configured to store the byproducts discharged through the byproduct outlet; a drain valve configured to adjust an opening degree of the drain part; and a controller configured to control operations of the pulverizing transfer part and the drain valve to perform: in a first mode, an operation of opening the drain valve and rotating the pulverizing transfer part in a first direction to grind and transfer the object introduced through the inlet; and in a second mode, an internal cleaning operation of operating the pulverizing transfer part in a state in which the drain valve is closed and water is received in the treatment chamber.

The treatment chamber may include a water inlet portion configured to allow externally supplied water to pass therethrough, where the food waste disposal device further includes a water supply valve configured to control an opening and a closing of the water inlet portion.

The controller may be further configured to perform, in the second mode, an operation of opening the water supply valve to supply water into the treatment chamber.

The food waste disposal device may further include an odor sensor configured to detect an odor signal indicating a concentration of malodorous components in the treatment chamber, where the controller is further configured to perform the second mode in a state in which the concentration of malodorous components in the treatment chamber is equal to or greater than a preset level.

The controller may be further configured to perform, in the second mode, an operation of at least partially opening the drain valve and rotating the pulverizing transfer part in the first direction after performing the internal cleaning operation.

The controller may be further configured to perform, in the second mode, an operation of alternately rotating the pulverizing transfer part in the first direction and a second direction opposite to the first direction during the internal cleaning operation.

The food waste disposal device may further include a strainer on the drain part and including a plurality of holes with a predetermined size.

The food waste disposal device may further include a gasket configured to close the byproduct outlet, and to open the byproduct outlet in a state in which a pressure is applied that is equal to or greater than a predetermined level.

The food waste disposal device may further include: a blower fan configured to dry the byproducts stored in the storage part; and a moisture exhaust pipe including a first end connected to an upper side of the storage part and a second end connected to the sink drainpipe.

According to an aspect of the disclosure, a food waste disposal device may include: a treatment chamber including a first chamber having an open area, a second chamber coupled to the open area and having a cross-sectional area that decreases away from the first chamber, and a byproduct outlet at an end portion of the second chamber away from the first chamber; and a pulverizing transfer part inside both the first chamber and the second chamber in the treatment chamber, and including a rotating body, a first blade extending from an outer circumferential surface of a portion of the rotating body in the first chamber, and a second blade extending from an outer circumferential surface of a portion of the rotating body positioned in the second chamber and having a shorter length than the first blade.

The first blade may be configured to rotate the rotating body to grind an object introduced into the treatment chamber, where the second blade is configured to compress and transfer the object ground by the rotating body to the byproduct outlet.

The rotating body may include a first body in the first chamber and a second body in the second chamber, where a thickness of the second body is larger than a thickness of the first body.

The food waste disposal may further include a gasket configured to close the byproduct outlet, and to open the byproduct outlet in a state in which a pressure is applied that is equal to or greater than a predetermined level.

The food waste disposal device may further include: a storage part configured to store byproducts discharged from the byproduct outlet; a blower fan configured to dry byproducts stored in the storage part; and a moisture exhaust pipe including a first end connected to an upper side of the storage part and configured to discharge moisture inside the storage part.

The second chamber may be conical or frustoconical in shape.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a view illustrating an overall structure of a food waste disposal device according to an embodiment;

FIG. 2 is a functional block diagram schematically illustrating a configuration of a food waste disposal device according to an embodiment in terms of function and control;

FIGS. 3A and 3B are views illustrating an operation in which a food waste disposal device treats food waste according to an embodiment;

FIG. 4 is a flowchart illustrating a control method for treating food waste by a food waste disposal device according to an embodiment;

FIGS. 5A, 5B, and 5C are views illustrating an operation in which a food waste disposal device cleans the inside, according to an embodiment; and

FIG. 6 is a flowchart illustrating a control method for treating food waste by a food waste disposal device according to an embodiment.

### DETAILED DESCRIPTION

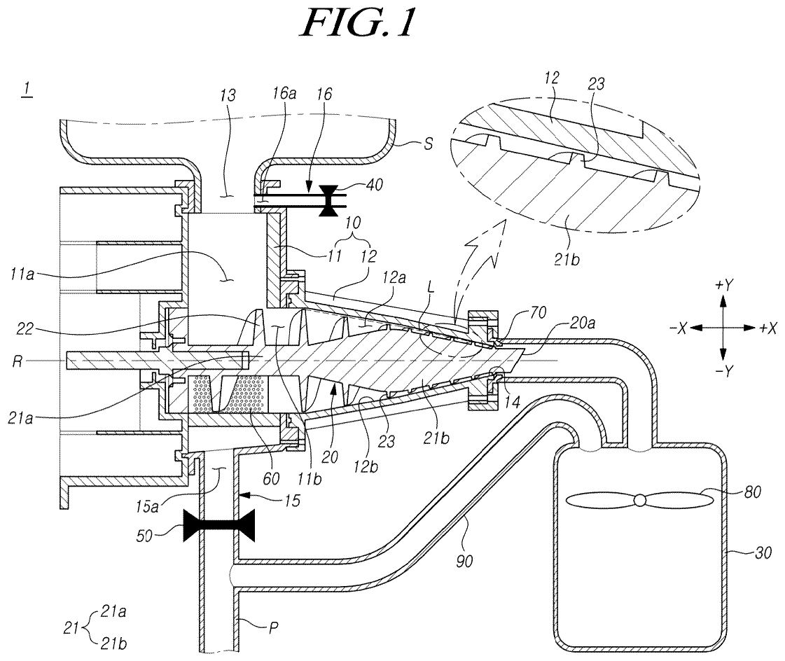

Various embodiments of the disclosure are merely exemplified herein with reference to FIGS. 1 to 6, to describe the principle of the disclosure, and should not be interpreted as limiting the scope of the disclosure. Those skilled in the art will understand that the principle of the disclosure may be implemented in any appropriately disposed system or device.

Hereinafter, embodiments of the disclosure are described in detail with reference to the drawings so that those skilled in the art to which the disclosure pertains may easily practice the disclosure. However, the disclosure may be implemented in other various forms and is not limited to the embodiments set forth herein. The same or similar reference denotations may be used to refer to the same or similar elements throughout the specification and the drawings. Further, for clarity and brevity, no description is made of well-known functions and configurations in the drawings and relevant descriptions.

FIG. 1 is a view illustrating an overall structure of a food waste disposal device according to an embodiment.

Referring to FIG. 1, the food waste disposal device 1 according to an embodiment may include at least one of a treatment chamber 10, a pulverizing transfer part 20, a storage part 30, a water supply valve 40, a drain valve 50, or a filter 60. The food waste disposal device 1 may be disposed to be connected to the sink S. The food waste disposal device 1 may be disposed under the sink S. An object (e.g., food waste) input from the sink S may be introduced into the treatment chamber 10, and may be ground, compressed, and transferred by the pulverizing transfer part 20. Byproducts of ground and compressed food waste may be discharged to the storage part 30 by the operation of the pulverizing transfer part 20. The byproduct discharged to the storage part 30 may have a predetermined amount of moisture, and for this purpose, a blower fan 80 forming an airflow may be disposed in the storage part 30. The moisture in the storage part 30 may be discharged to the drainpipe P through the moisture exhaust pipe 90 by the blower fan 80. The liquid or fine material in the treatment chamber 10 may be filtered by the filter 60 and then discharged to the drainpipe P through the drain part 15.

According to an embodiment, the treatment chamber 10 may be disposed to be connected to the sink S. The treatment chamber 10 may be directly or indirectly connected to the sink S. For example, a structure such as a connection pipe for connecting the treatment chamber 10 and the sink S may be provided therebetween.

According to an embodiment, the treatment chamber 10 may include a first chamber 11 and a second chamber 12. The first chamber 11 may have an open area 11b in a portion thereof. The second chamber 12 may be coupled to the open area 11b to have a cross-sectional area decreasing away from the first chamber 11. As an example, the second chamber 12 may have a conical or frustoconical shape. The first chamber 11 and the second chamber 12 may be combined to form a space for receiving the pulverizing transfer part 20 therein. The first chamber 11 and the second chamber 12, as separate components, may be combined with each other to form the treatment chamber 10, but the disclosure is not limited thereto, and the first chamber 11 and the second chamber 12 may be integrally formed.

According to an example, the first chamber 11 may form a first space 11a therein, and the second chamber 12 may form a second space 12a therein. The first space 11a and the second space 12a may be components divided for each area in a single connected space. The volume of the first space 11a may be larger than the volume of the second space 12a, but the disclosure is not limited thereto.

According to an embodiment, the treatment chamber 10 may include an inlet 13 connected to the sink S. The material discharged from the sink S may be introduced into the treatment chamber 10 through the inlet 13. The inlet 13 may be positioned in the upper side of the treatment chamber 10, e.g., but the disclosure is not limited thereto. For example, the inlet 13 may be positioned in the upper side of the first chamber 11. The food waste introduced through the inlet 13 may be ground in a portion of the first space 11a of the first chamber 11 and the second space 12a of the second chamber 12.

According to an example, the treatment chamber 10 may be disposed to be connected to the drainpipe P. The treatment chamber 10 may include a drain part 15 connected to the drainpipe P. A liquid or a small-sized material in the treatment chamber 10 may be discharged through the drain opening 15a provided in the drain part 15. The drain part 15 may have, e.g., a pipe shape having a predetermined length. However, the disclosure is not limited thereto, and the drain part 15 may have various shapes with a passage provided therein. The drain part 15 may be composed of only the drain opening 15a, for example.

According to an embodiment, the drain part 15 may be positioned on the lower side of the first chamber 11. The liquid introduced through the inlet 13 or the liquid or fine material generated during the grinding process may be discharged to the drainpipe P through the drain part 15.

According to an example, the treatment chamber 10 may include a byproduct outlet 14 for discharging byproducts ground and compressed in the chamber. The food waste ground by the rotational operation of the pulverizing transfer part 20, which is described below, may be transferred to the byproduct outlet 14. The ground food waste becomes a byproduct and is transferred to the byproduct outlet 14 positioned at the end portion of the second chamber 12 and, while transferred, the byproduct may be compressed in the second space 12a. The byproduct outlet 14 may be positioned at an end portion of the second space 12a of the treatment chamber 10. The byproduct outlet 14 may be positioned, e.g., at the end portion of the second space 12a away from the first space 11a. The byproduct outlet 14 may be, e.g., disposed adjacent to the free end 20a of the pulverizing transfer part 20.

According to an embodiment, the food waste disposal device 1 may further include a gasket 70 disposed to close the byproduct outlet 14. The gasket 70 may be provided to be opened when a pressure of a predetermined level or more is applied. The gasket 70 may be formed of, e.g., an elastic material. When the pressure larger than or equal to the predetermined level is released, the gasket 70 may close the byproduct outlet 14 again. For example, food waste (or byproducts) compressed by the pulverizing transfer part 20 may pass through the byproduct outlet 14 by pushing the gasket 70.

According to an embodiment, a strainer 60 may be disposed on the drain part 15. The strainer 60 may have, e.g., several holes with a predetermined size. The material larger than the size of the hole may be filtered by the strainer 60. The strainer 60 filters out material larger than a predetermined size, allowing only material or liquid smaller than this size to pass through and be drained into the drain part 15.

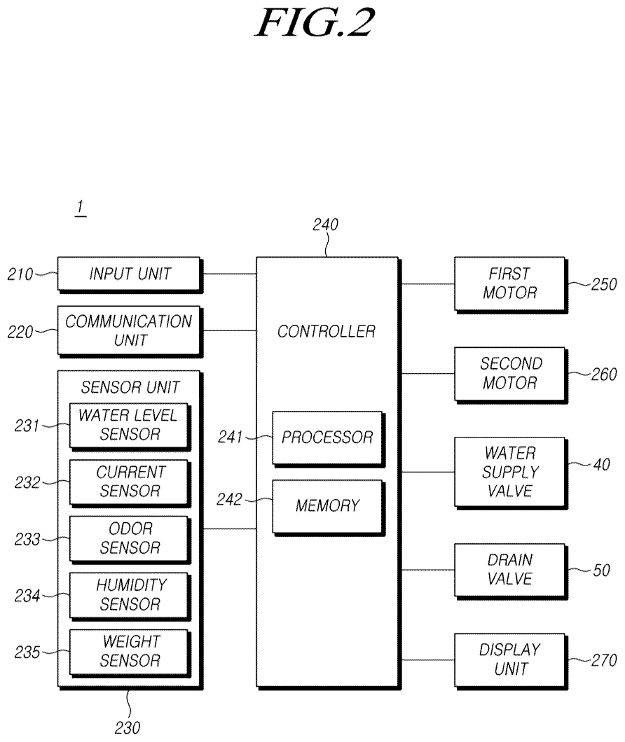

According to an example, the pulverizing transfer part 20 may be disposed to be rotatable in the treatment chamber 10. The pulverizing transfer part 20 may be rotated using the length direction of the pulverizing transfer part 20 as the rotation axis R. The pulverizing transfer part 20 may be rotated by driving a grinding motor (e.g., the first motor 250 of FIG. 2) coupled to the pulverizing transfer part 20. The pulverizing transfer part 20 may have a screw shape as a whole, but the disclosure is not limited thereto. The blade portion of the pulverizing transfer part 20 may have a length that varies according to the position, for example. For example, the blades 22 and 23 of the grinding and transfer portion 20 may have a length decreasing toward the free end 20a of the grinding and transfer portion 20, but the disclosure is not limited thereto. The free end 20a of the grinding and transfer portion 20 may be disposed to penetrate the byproduct outlet 14, but the disclosure is not limited thereto.

According to an example, the pulverizing transfer part 20 may include at least one of a rotating body 21, a first blade 22, and a second blade 23. The rotating body 21 may be coupled to the first motor 250. For example, the rotating body 21 may be coupled to the shaft of the first motor 250 to receive rotational power from the first motor 250. The pulverizing transfer part 20 may be rotated using the central axis of the rotating body 21 in the length direction as the rotation axis R.

The first blade 22 or the second blade 23 may extend from the outer circumferential surface of the rotating body 21. For example, the first blade 22 may extend from the outer circumferential surface of a portion of the rotating body 21, and the second blade 23 may extend from the outer circumferential surface of the rest of the rotating body 21. The first blade 22 or the second blade 23 may extend in a spiral shape, e.g., but the disclosure is not limited thereto. When the rotating body 21 is rotated, the first blade 22 and the second blade 23 around the rotating body 21 are rotated to be involved in grinding, compressing, and transferring food waste. If the rotating body 21 rotates, the first blade 22 may mainly grind food waste. For example, if the rotating body 21 rotates, the second blade 23 may compress and transfer food waste that is mainly ground into small pieces by the first blade 22. If the rotating body 21 rotates, the second blade 23 may discharge the compressed food waste to the byproduct outlet 14.

According to an example, the pulverizing transfer part 20 may be disposed in the treatment chamber 10. The pulverizing transfer part 20 may be disposed inside the first chamber 11 and the second chamber 12, for example. For example, the rotating body 21 may be disposed inside the first chamber 11 and the second chamber 12 of the treatment chamber 10. The first blade 22 may be positioned in the first chamber 11. The first blade 22 may be positioned not only in the first chamber 11 but also in the second chamber 12 adjacent to the first chamber 11. The second blade 23 may be positioned in the second chamber 12.

According to an embodiment, the rotating body 21 may include a first body 21a and a second body 21b. One end of the first body 21a may be coupled to the grinding motor (e.g., the first motor 250 of FIG. 2). The second body 21b may extend from the other end of the first body 21a. The first blade 22 may extend from the outer circumferential surface of the first body 21a. The second blade 23 may extend from the outer circumferential surface of the second body 21b. The first body 21a may be positioned in the first chamber 11, for example. The second body 21b may be positioned in the second chamber 12, for example. The first body 21a and the second body 21b may be integrally formed as illustrated, but the disclosure is not limited thereto and may be coupled to each other as separate components.

According to an example, the thickness of the second body 21b may be larger than the thickness of the first body 21a. Here, the thickness may refer to an average thickness. For example, the maximum thickness of the second body 21b may be larger than the maximum thickness of the first body 21a. The second body 21b may have, e.g., a shape in which the outer circumferential surface protrudes convexly, but the disclosure is not limited thereto. The second body 21b occupies most of the second space 12a in the second chamber 12, and only food waste that is ground into pieces smaller than a predetermined size may be transferred to the space between the second chamber 12 and the second body 21b.

According to an example, the length of the first blade 22 may be larger than the length of the second blade 23. Here, the length of the first blade 22 and the length of the second blade 23 may refer to a length protruding radially outward of the rotating body 21 from the rotating body 21. The first blade 22 may be formed to grind food waste according to the rotation of the rotating body 21. The second blade 23 may be formed to compress and transfer food waste ground by the first blade 22. As described above, in the food waste disposal device 1 according to an embodiment of the disclosure, since the pulverizing transfer part 20 performs not only grinding but also compression and transfer of byproducts, the number of the components of the food waste disposal device 1 may be reduced, resulting in a reduction in manufacturing cost and volume.

According to an example, the virtual straight line L connecting the end portions of the second blade 23 in the length direction of the rotating body 21 may be formed to be inclined so as to be closer to the rotating axis R toward the byproduct outlet 14. The virtual straight line L may correspond to the shape of the second chamber 12.

According to an embodiment, an end portion of the second blade 23 may be adjacent to the inner wall 12b of the second chamber 12. Therefore, as the distance between the inner wall 12b of the second chamber 12 and the outer circumferential surface of the second body 21b decreases, the length of the second blade 23 may decrease.

According to an example, the storage part 30 may be configured to store byproducts discharged from the byproduct outlet 14 of the treatment chamber 10. The storage part 30 may store byproducts by forming an internal empty space. The storage part 30 may be provided to be detachable. The user may separate the storage part 30 to remove byproducts stored therein.

According to an example, the food waste disposal device 1 may further include a blower fan 80 for drying the inside of the storage part 30. The blower fan 80 may be provided to form airflow in the inner space of the storage part 30. The blower fan 80 may be rotated by driving a blowing motor (e.g., the second motor 260 of FIG. 2) coupled to the rotation shaft of the blower fan 80. Moisture inside the storage part 30 may be removed or byproducts may be dried due to the airflow formed by the blower fan 80.

According to an example, the food waste disposal device 1 may further include a moisture exhaust pipe 90 having one end connected to the storage part 30. The moisture exhaust pipe 90 may have one end connected to the upper side of the storage part 30, for example. The other end of the moisture exhaust pipe 90 may be connected to the drainpipe P. The airflow formed by the blower fan 80 may pass through the moisture exhaust pipe 90 and be transferred to the drainpipe P. For example, moisture or water vapor of byproducts remaining inside the storage part 30 may be discharged to the drainpipe P through the moisture exhaust pipe 90.

According to an embodiment, the drain valve 50 may be configured to adjust the degree of opening of the drain part 15. The opening and closing of the drain valve 50 may be controlled by a controller (e.g., the controller 240 of FIG. 2). The drain valve 50 may be, e.g., a solenoid valve, a ball valve, a check valve, a gate valve, a glove valve, or a butterfly valve, but the disclosure is not limited thereto.

According to an embodiment, the treatment chamber 10 may include a water inlet portion 16. The water inlet portion 16 may be disposed above the treatment chamber 10. The water inlet portion 16 may be a passage through which water is supplied from the outside by being connected to a separate water supply pipe. The water inlet portion 16 may include a water supply opening 16a. Water may be supplied to the treatment chamber 10 through the water supply opening 16a. The water inlet portion 16 may have, e.g., a pipe shape having a predetermined length. However, the disclosure is not limited thereto, and the water inlet portion 16 may have various shapes having a passage provided therein. The water inlet portion 16 may be composed of only the water supply opening 16a, for example.

According to an example, the water supply valve 40 may be configured to control the opening and closing of the water inlet portion 16 provided to supply water to the treatment chamber 10. The opening and closing of the water supply valve 40 may be controlled by a controller (e.g., the controller 240 of FIG. 2). The water supply valve 40 may be, e.g., a solenoid valve, a ball valve, a check valve, a gate valve, a glove valve, or a butterfly valve, but the disclosure is not limited thereto.

FIG. 2 is a functional block diagram schematically illustrating a configuration of a food waste disposal device according to an embodiment in terms of function and control.

The food waste disposal device 1 may include an input unit 210. The input unit 210 may include any type of user input means for obtaining setting information from the user for controlling the operation of the food waste disposal device 1. Various user inputs obtained through the input unit 210 may be transferred to the controller 240 to be described below. In an example, various user inputs obtained through the input unit 210 may be transmitted to the outside through the communication unit 220 to be described below, but the disclosure is not limited thereto.

In an example, the food waste disposal device 1 may include a communication unit 220 that supports signal transmission/reception to/from the outside. In an example, the communication unit 220 may receive and/or transmit a wired/wireless signal to/from an external wired/wireless communication system, an external server, and/or other devices according to a predetermined wired/wireless communication protocol. In an example, the communication unit 220 may include one or more modules to connect the food waste disposal device 1 to one or more networks. In an example, the communication unit 220 may include at least one of a mobile communication module, a wired/wireless Internet module, a short-range communication module, and/or a location information module.

In an example, the mobile communication module may transmit/receive wireless signals with at least one of an external bracket structure, an external UE, and an external server through the mobile communication network according to any communication protocol among various communication protocols for mobile communication. The wireless signals may include various types of data signals. In an example, the wireless signals may include voice call signals, video call signals, and text/multimedia message signals, but the disclosure is not limited thereto.

For example, the wired/wireless Internet module may support wireless LAN (WLAN), wireless-fidelity (Wi-Fi), Wi-Fi direct, digital living network alliance (DLNA), wireless broadband (WiBro), world interoperability for microwave access (WiMAX), high speed downlink packet access (HSDPA), high speed uplink packet access (HSUPA), long term evolution (LTE), or long term evolution-advanced (LTE-A), but is not limited thereto. In an example, the wired/wireless Internet module of the communication unit 220 may transmit/receive data according to at least one wired/wireless Internet technology among Internet technologies not listed above.

The short-range communication module may be intended for, e.g., short-range communication and may support short-range communication using at least one of Bluetooth, radio frequency identification (RFID), infrared data association (IrDA), ultra-wideband (UWB), ZigBee, near-field communication (NFC), Wi-Fi, Wi-Fi Direct, or wireless universal serial bus (USB) technology. The short-range communication module may support, e.g., wireless communication between the food waste disposal device 1 and a wireless communication system, between the food waste disposal device 1 and another device, or between the food waste disposal device 1 and a network in which the other device is positioned through a short-range wireless communication network.

The location information module may be, e.g., a global positioning system (GPS) module or a Wi-Fi module as a module for obtaining the location of the food waste disposal device 1. When the food waste disposal device 1 utilizes the GPS module, the food waste disposal device 1 may receive information about the location of the food waste disposal device 1 using a signal transmitted from the GPS satellite. When the food waste disposal device 1 utilizes the Wi-Fi module, the food waste disposal device 1 may receive information about the location of the food waste disposal device 1 based on information about a wireless access point (AP) that transmits and receives a wireless signal to and from the Wi-Fi module.

In an example, the communication unit 220 may receive the configuration data signal input by the user on the mobile terminal of the user in the form of a wireless signal according to a predetermined wireless communication protocol. In an example, the communication unit 220 may receive information and/or a command for controlling the operation of the food waste disposal device 1 from an external server in the form of a signal according to a predetermined wired/wireless communication protocol. The communication unit 220 may transfer various received signals to the controller 240 to be described below. In an example, the communication unit 220 may transmit various data generated or obtained on the food waste disposal device 1 in the form of a wired/wireless signal according to a predetermined wired/wireless communication protocol, e.g., to a mobile terminal of the user or an external server.

According to an embodiment, the food waste disposal device 1 may include a sensor unit 230 for detecting an operating state and/or an internal environment of the food waste disposal device 1. In an example, the sensor unit 230 may include at least one of a water level sensor 231, a current sensor 232, a humidity sensor 234, an odor sensor 233, or a weight sensor 235, but this is exemplary and the disclosure is not limited thereto.

According to an example, the water level sensor 231 is a sensor provided to detect the water level in the treatment chamber 10. For example, the water level sensor 231 may be positioned on the lower side of the treatment chamber 10 to detect a pressure difference according to the water level of water introduced into the treatment chamber 10 as an electrical signal. In an example, the water level sensor 231 may be provided to determine the water level in a water supply circumstance for the controller 240 to clean the inside of the treatment chamber 10. In an example, the water level sensor 231 may transfer an electrical signal regarding the water level in the treatment chamber 10 to the controller 240.

According to an example, the current sensor 232 may be provided to detect a current flowing through the first motor 250 for rotating the pulverizing transfer part 20. The electrical signal regarding the current value of the first motor 250 generated by the current sensor 232 may be transferred to the controller 240. The current sensor 232 may be provided to detect a current load applied to the first motor 250. When the current sensor 232 detects a high current when the first motor 250 rotates, the controller 240, which is described below, may determine that food waste that puts a load on the rotation of the pulverizing transfer part 20, remains therein. According to an embodiment, the odor sensor 233 may be provided to detect an odor signal indicative of concentration of malodorous components in the treatment chamber 10. The odor sensor 233 may be disposed in the treatment chamber 10. The odor sensor 233 may detect odor by measuring the concentration of volatile organic compound (VOC) present in the air in the treatment chamber 10. The odor sensor 233 may detect the degree of odor by measuring the concentration of the volatile organic compound.

According to an embodiment, the humidity sensor 234 may be provided to detect humidity in the storage part 30. The humidity sensor 234 may be disposed in the storage part 30. The humidity sensor 234 may detect the amount of water vapor in the atmosphere of the storage part 30.

According to an example, the weight sensor 235 may be provided to detect the weight of the byproduct stored in the storage part 30. The weight sensor 235 may be provided at the lower portion of the storage part 30. By measuring the weight of the storage part 30 by the weight sensor 235, the amount of byproducts accumulated in the storage part 30 may be estimated.

Each of the water level sensor 231, the current sensor 232, the odor sensor 233, the humidity sensor 234, and/or the weight sensor 235 of the disclosure may include an electrically connected sensor circuit unit. The sensor circuit unit may be electrically connected to the processing circuit unit of the processor 241, or may be included as at least a portion of the processing circuit unit.

According to an example, the food waste disposal device 1 may include a controller 240 that controls the overall operation of the food waste disposal device 1. The controller 240 may include memory 242 for storing a program and/or data for controlling each component of the food waste disposal device 1, and a processor 241 for generating a control signal for controlling each component of the food waste disposal device 1 according to the program and/or data stored in the memory 242, and information obtained from each of the other components.

According to an example, the controller 240 may include at least one of circuits such as a central processing unit (CPU), microprocessor unit (MPU), graphics processing unit (GPU), accelerated processing unit (APU), digital signal processor (DSP), field-programmable gate array (FPGA), control processor (CP), application processor (AP), system on chip (soc), or integrated circuit (IC).

According to an example, the memory 242 may store various data that may be used to control the operation of each component of the food waste disposal device 1. The memory 242 may store, e.g., the plurality of application programs used in the food waste disposal device 1, data for controlling the operation of the food waste disposal device 1, and instructions. At least some of the application programs stored in the memory 242 may be downloaded from an external server through wireless communication. At least some of the application programs stored in the memory 242 may be stored in the memory 242 from the time of shipment for default functions of the food waste disposal device 1.

In an example, the processor 241 of the controller 240 may receive various input/setting information, such as power on/off of the food waste disposal device 1, operation setting information of the food waste disposal device (e.g., operation start/stop, food waste disposal mode selection, cleaning mode selection, operation time selection, etc.), or other various control information from the above-described input unit 210 and/or communication unit 220. The processor 241 may obtain various sensing information from the sensor unit 230, such as information about the water level in the treatment chamber 10 detected by the water level sensor 231, information about the current flowing in the first motor 250 detected by the current sensor 232, information about the odor inside the treatment chamber 10 detected by the odor sensor 233, and information about the humidity in the storage part 30 detected by the humidity sensor 234.

In an example, the processor 241 of the controller 240 may generate an operation control command for each component of the food waste disposal device 1 based on various information received from the input unit 210, the communication unit 220, and/or the sensor unit 230. In an example, the processor 241 may control each related component to perform the operation mode input to the input unit 210. In an example, the processor 241 may adjust the water supply amount during the operation of the internal cleaning mode to be described below using the information about the water level of the treatment chamber 10 received from the water level sensor 231. In an example, the processor 241 may control the operation of the first motor 250 by determining whether a material to be ground, compressed, and transferred remains in the treatment chamber 10 using information about the current load of the first motor 250 received from the current sensor 232. In an example, the processor 241 may control at least one of the water supply valve 40, the first motor 250, or the drain valve 50 by determining whether internal cleaning of the treatment chamber 10 is required using information about the amount of gas in the treatment chamber 10 received from the odor sensor 233. In an example, the processor 241 may control the operation of the second motor 260 rotating the blower fan 80 using information about the humidity in the storage part 30 received from the humidity sensor 234. In an example, the processor 241 may transfer information indicating that the byproduct should be emptied to the user through the display unit 270 or the communication unit 220 using information about the amount of byproducts accumulated in the storage part 30 received from the weight sensor 235.

The processor 241 may control the operation of at least one of the first motor 250, the water supply valve 40, the drain valve 50, or the second motor 260 to control the execution of the food waste disposal mode (e.g., the first mode to be described below) or the internal cleaning mode (e.g., the second mode to be described below) of the food waste disposal device 1.

According to an embodiment, the first motor 250 may include a first motor control circuit unit. The first motor control circuit unit may control the operating intensity or rotational speed of the first motor 250. The first motor control circuit unit may be electrically connected to the processing circuit unit of the processor 241, or may be configured as at least a portion of the processing circuit unit.

According to an example, the second motor 260 may include a second motor control circuit unit. The second motor control circuit unit may control the operating intensity or rotational speed of the second motor 260. The second motor control circuit unit may be electrically connected to the processing circuit unit of the processor 241 or may be configured as at least a portion of the processing circuit unit.

In an example, the processor 241 may control the driving of the first motor 250 to rotate the pulverizing transfer part 20. In an example, the processor 241 may control the opening and closing of the water supply valve 40 to adjust the amount of water supplied to the treatment chamber 10. In an example, the processor 241 may control the opening and closing of the drain valve 50 so that liquid or fine materials in the treatment chamber 10 are discharged to the drainpipe P. In an example, the processor 241 may control driving of the second motor 260 to rotate the blower fan 80. The processor 241 may continuously obtain information from the input unit 210, the communication unit 220, and/or the sensor unit 230 even while the control operation is performed, and may continuously update and control the operation of each component based on the obtained information.

In an example, the processor 241 of the controller 240 may generate a command for controlling whether and how to display information through the display unit 270, based on various types of information received from the input unit 210, the communication unit 220, and/or the sensor unit 230.

In an example, the processor 241 of the controller 240 may perform a first mode or a second mode for operating the food waste disposal device 1. The first mode may be, e.g., a mode for performing the operation for grinding, compressing, and transferring food waste introduced into the treatment chamber 10. The first mode may be referred to as a food waste disposal mode. The second mode may be, e.g., a mode for performing the operation for cleaning the inside of the treatment chamber 10. The second mode may be referred to as an internal cleaning mode. Information about the operation according to the first mode or the operation according to the second mode may be previously stored in the memory 242.

According to an example, the processor 241 may include a processing circuit unit. The processing circuit unit may be electrically connected to the communication unit 220. The processing circuit unit may be electrically connected to the sensor circuit unit or may include the sensor circuit unit. The processing circuit unit may be electrically connected to the first motor control circuit unit or may include the first motor control circuit unit. The processing circuit unit may be electrically connected to the second motor control circuit unit or may include the second motor control circuit unit.

The operation mode of the food waste disposal device 1 may be set automatically or manually. For example, the user may set the operation mode of the food waste disposal device 1 through an input of the input unit 210. For example, the user may set the operation mode of the food waste disposal device 1 using an electronic device such as a mobile phone terminal connected to the communication unit of the food waste disposal device 1. For example, the processor 241 of the controller 240 may automatically perform the first mode by detecting that food waste is introduced therein. For example, the processor 241 of the controller 240 may perform the second mode when determining that a predetermined level of odor has occurred in the treatment chamber 10 using the odor sensor 233. The methods for operating the food waste disposal device 1 described above are exemplary and are not limited thereto.

In the drawings, the controller 240 is disclosed as one comprehensive component that controls all of the components included in the food waste disposal device 1, but the disclosure is not limited thereto. In an example, the food waste disposal device 1 may be configured to include a plurality of controller components that individually control some of the components of the food waste disposal device 1.

In an example, the food waste disposal device 1 may include, e.g., a separate controller including a processor and memory for controlling the operation of the first motor 250. In an example, the food waste disposal device 1 may include a separate controller including a processor and memory for controlling the operation of the user interface according to a user input. The processor 241 of the controller 240 may include a plurality of processors, and the memory 242 may include a plurality of memory devices. The processor 241 may include a circuit. The memory 242 may include a circuit. The processor 241 and the memory 242 may be integrally formed.

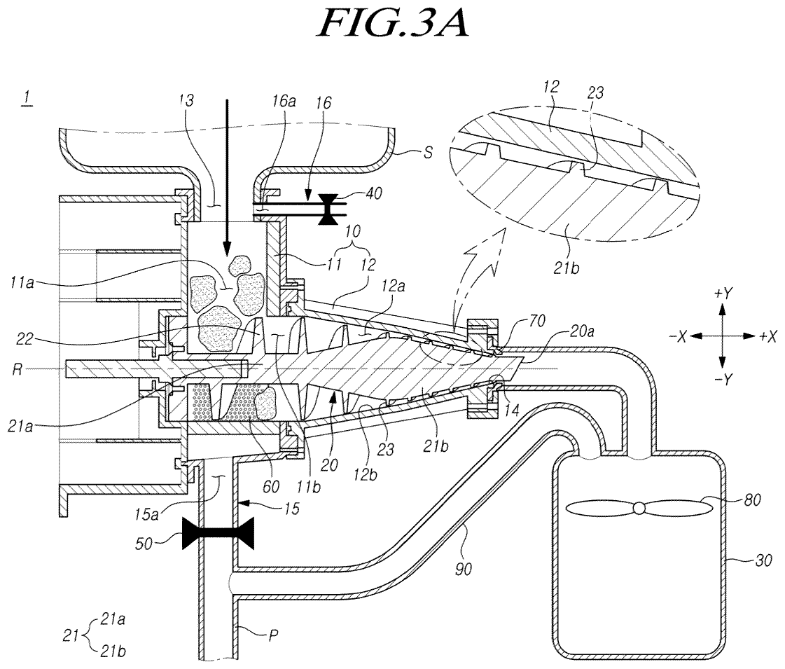

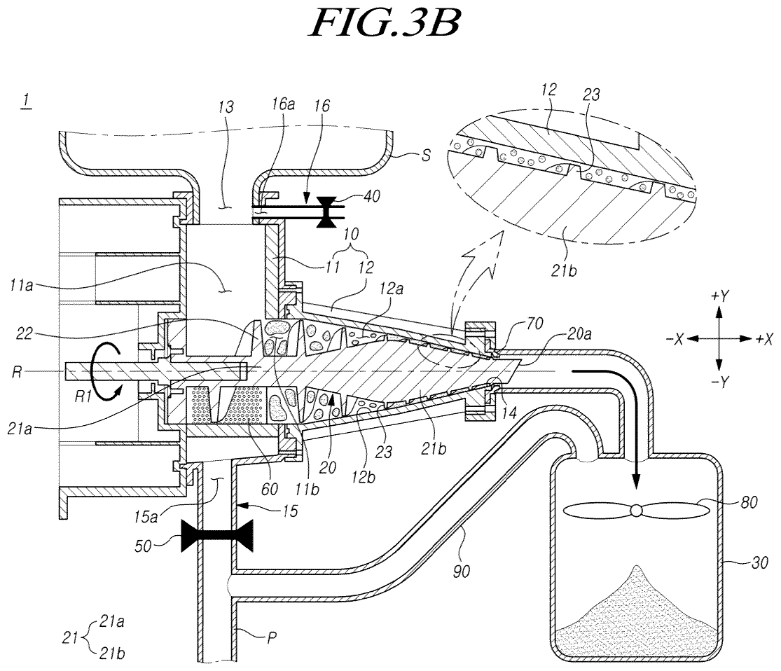

FIGS. 3A and 3B are views illustrating an operation in which a food waste disposal device treats food waste according to an embodiment.

FIGS. 3A and 3B are example views for describing a process of performing the first mode (e.g., the food waste disposal mode) operation described in FIG. 2 by the food waste disposal device 1. The portions that overlap the above-described content are omitted hereinafter.

Referring to FIG. 3A, various materials including food waste may be introduced into the treatment chamber 10 of the food waste disposal device 1. Liquids such as water introduced together with food waste may be discharged to the drainpipe P through the strainer 60. In this case, the drain valve 50 may be in an open state. Materials not filtered by the strainer 60 may be positioned mainly in the first chamber 11 of the treatment chamber 10.

Referring to FIG. 3B, the controller 240 may control the operation of the first motor 250 to rotate the pulverizing transfer part 20. The controller 240 may rotate the first motor 250 in the first direction R1 so that the pulverizing transfer part 20 rotates in the first direction R1 (or forward direction). If the pulverizing transfer part 20 is rotated in the first direction R1, the food waste may be transferred in the direction toward the byproduct outlet 14 by the spiral shape of the first blade 22 and the second blade 23. For example, if the pulverizing transfer part 20 rotates in the first direction R1, the food waste may be transferred in the +X direction of FIG. 3B.

If the pulverizing transfer part 20 is rotated, the food introduced into the treatment chamber 10 may be finely ground. The ground food waste may be transferred to the second chamber 12 in the spiral direction of the first blade 22 and the second blade 23. The ground food waste transferred to the second chamber 12 may be compressed by rotation of the second blade 23 and discharged to the byproduct outlet 14. While the ground food waste is compressed in the second chamber 12, moisture included in the ground food waste may be removed.

In an example, for smooth grinding of food waste introduced into the treatment chamber 10, the controller 240 may open the water supply valve 40 to supply water into the treatment chamber 10.

The ground and compressed food waste (hereinafter, referred to as byproducts) that has passed through the byproduct outlet 14 may be transferred to the storage part 30 and stored. Since these byproducts still include some moisture, the controller 240 may operate the blower fan 80 in the storage part 30 to dry them. The water vapor formed by the blower fan 80 may pass through the moisture exhaust pipe 90 and be discharged to the drainpipe P.

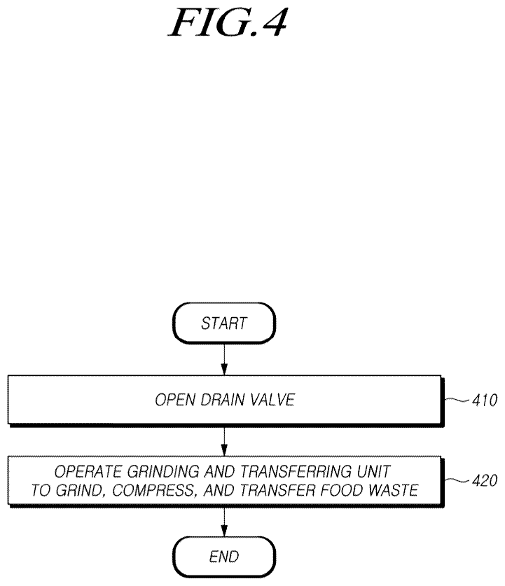

FIG. 4 is a flowchart illustrating a control method for treating food waste by a food waste disposal device according to an embodiment.

FIG. 4 is a flowchart for describing a control process of a first mode (e.g., a food waste disposal mode) operation of the food waste disposal device 1 illustrated in FIGS. 3A and 3B. FIG. 4 illustrates a control process in which the first mode is executed in a state in which food waste is introduced to process (grinding, compressing, and transferring) food waste. The portions that overlap the above-described content are omitted hereinafter.

According to an example, the controller (e.g., the controller 240 of FIG. 2) of the food waste disposal device (e.g., the food waste disposal device 1 of FIG. 1) may open the drain valve 50 in operation 410. The drain valve 50 may be opened to allow liquids or fine materials that may pass through the strainer (e.g., the strainer 60 of FIG. 1) among the materials introduced from the inlet (e.g., the inlet 13 of FIG. 1) of the treatment chamber (e.g., the treatment chamber 10 of FIG. 1) to pass through the drain part (e.g., the drain part 15 of FIG. 1) and be discharged to the sewer.

According to an example, in operation 420, the controller 240 may grind, compress, and transfer food waste by operating the pulverizing transfer part (e.g., the pulverizing transfer part 20 of FIG. 1). The controller 240 may drive the grinding motor (e.g., the first motor 250 of FIG. 2) to rotate the pulverizing transfer part 20. The controller 240 may determine the degree of grinding progress of the food waste using a current sensor (e.g., the current sensor 232 of FIG. 2) for detecting the current of the first motor 250 while rotating the pulverizing transfer part 20.

For example, when the controller 240 determines that the grinding, compression, and transfer of food waste is completed using the current sensor 232, the controller 240 may terminate the first mode. For example, the controller 240 may rotate the pulverizing transfer part 20 for a preset time to grind, compress, and transfer food waste. For example, the controller 240 may determine a time for rotating the pulverizing transfer part 20 according to a user input. A method for determining the time for rotating the pulverizing transfer part 20 in operation 420 is not limited to the above-described method.

According to an example, the controller 240 may open the water supply valve 40 to smoothly grind food waste while operation 420 is performed so that water flows into the treatment chamber 10. In an example, the controller 240 may open the water supply valve 40 in advance before operation 420 is performed so that water flows into the treatment chamber 10.

According to an example, the byproducts transferred in operation 420 may be transferred to the storage part 30. Since the byproducts transferred to the storage part 30 requires additional drying, the controller 240 may dry the inside of the storage part 30 by rotating the blowing motor (e.g., the second motor 260 of FIG. 2). The controller 240 may determine the degree of internal drying using, e.g., a humidity sensor (e.g., the humidity sensor 234 of FIG. 2) inside the storage part 30. The controller 240 may rotate the blower fan 80 when the humidity inside the storage part 30 detected by the humidity sensor 234 is larger than or equal to a predetermined value.

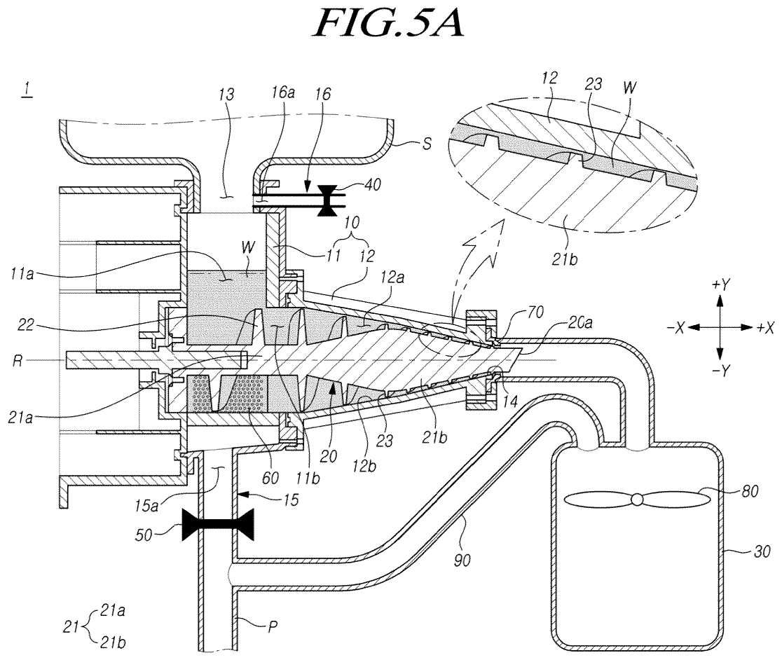

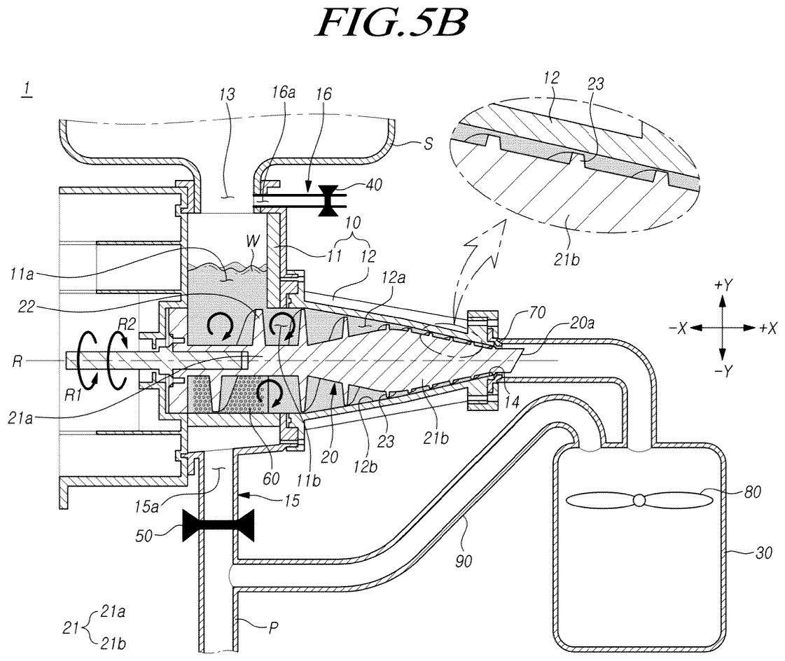

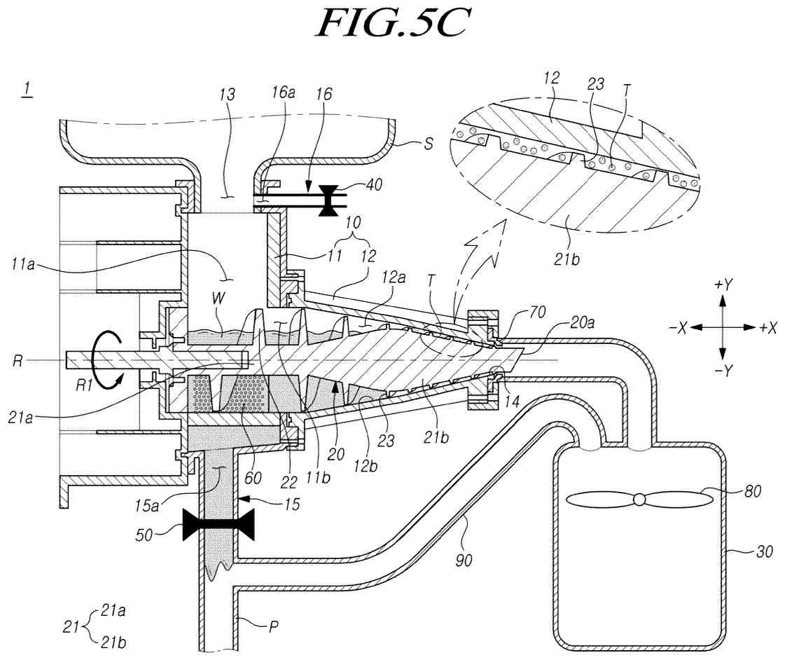

FIGS. 5A to 5C are views illustrating an operation in which a food waste disposal device cleans the inside, according to an embodiment.

FIGS. 5A, 5B, and 5C are example views for describing a process of performing the second mode (e.g., the internal cleaning mode) operation described in FIG. 2 by the food waste disposal device 1. The portions that overlap the above-described content are omitted hereinafter.

Referring to FIG. 5A, if the second mode is executed, the food waste disposal device 1 may first perform the operation of filling the treatment chamber 10 with water W. The controller 240 may open the water supply valve 40 to supply water to the treatment chamber 10. The controller 240 may close the drain valve 50 so that the water W in the treatment chamber 10 is not drained but is received in the treatment chamber 10. Since the byproduct outlet 14 is sealed by the gasket 70, water accumulated in the treatment chamber 10 may not flow to the storage part 30.

The controller 240 may measure the water level of the water W received in the treatment chamber 10 using the water level sensor 231. If it is determined that the water W fills the treatment chamber 10 by a preset water level, the controller 240 may close the water supply valve 40 to complete the water supply operation.

The configuration of the water inlet portion 16 and the water supply valve 40 may be omitted in the food waste disposal device 1 according to an embodiment. In this case, while the drain valve 50 is closed, the user may fill the treatment chamber 10 with water through a hose in the sink.

Referring to FIG. 5B, after water W fills the treatment chamber 10, the controller 240 may operate the first motor 250 to rotate the pulverizing transfer part 20. If the pulverizing transfer part 20 rotates, a water flow may be formed. Due to the water flow formed by the rotation of the pulverizing transfer part 20, it is possible to wash way food waste adhered to the inner wall of the treatment chamber 10 or the surface of the pulverizing transfer part 20.

In an example, the controller 240 may clean the inside of the treatment chamber 10 by the water flow formed by rotating the pulverizing transfer part 20 in the first direction R1 (e.g., forward direction) or the second direction R2 (e.g., reverse direction). For example, the controller 240 may perform internal cleaning by alternately rotating the pulverizing transfer part 20 in the first direction R1 and the second direction R2.

Referring to FIG. 5C, after internal cleaning by water flow formation is completed, the controller 240 may perform the control of opening the drain valve 50 while rotating the pulverizing transfer part 20 in the first direction R1. While water and the fine material that may pass through the strainer 60 are drained to the drain part 15, the food waste T remaining in the water after being washed by rotation of the pulverizing transfer part 20 in the first direction R1 may be transferred to the byproduct outlet 14 and discharged to the storage part 30. The controller 240 may open only a portion of the drain valve 50 to adjust the time during which the water in the treatment chamber 10 is discharged. If all the water is drained before the food waste T is transferred to the byproduct outlet 14, the food waste may be caught on the strainer 60, causing odor. Since the food waste T remaining in the water after the internal cleaning of the treatment chamber 10 should be transferred to the byproduct outlet 14, the controller 240 may adjust the rate at which the water W is drained by adjusting the degree of opening of the drain valve 50.

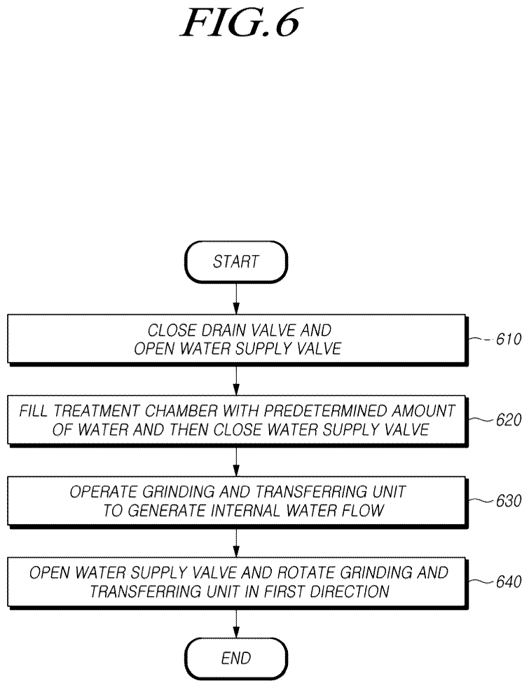

FIG. 6 is a flowchart illustrating a control method for treating food waste by a food waste disposal device according to an embodiment.

FIG. 6 is a flowchart for describing a process of controlling the second mode operation of the food waste disposal device 1 illustrated in FIGS. 5A, 5B, and 5C. The portions that overlap the above-described content are omitted hereinafter.

According to an example, the controller (e.g., the controller 240 of FIG. 2) of the food waste disposal device (e.g., the food waste disposal device 1 of FIG. 1) may perform the control to close the drain valve (e.g., the drain valve 50 of FIG. 1) and open the water supply valve (e.g., the water supply valve 40 of FIG. 1) in operation 610. While the drain valve 50 is closed, water supplied to the treatment chamber 10 by the water inlet portion (e.g., the water inlet portion 16 of FIG. 1) may be accumulated without being drained. Since the byproduct outlet (e.g., the byproduct outlet 14 of FIG. 1) is sealed by a gasket (e.g., the gasket 70 of FIG. 1), water accumulated in the treatment chamber 10 may not flow to the storage part (e.g., the storage part 30 of FIG. 1).

According to an example, in operation 620, the controller 240 may perform the control of closing the water supply valve 40 after filling the inside of the treatment chamber (e.g., the treatment chamber 10 of FIG. 1) with a predetermined amount of water. For example, the controller 240 may measure the amount of water included in the treatment chamber 10 using a water level sensor (e.g., the water level sensor 231 of FIG. 2). For example, the controller 240 may estimate the water level in the treatment chamber 10 using the time when the water supply valve 40 is opened. For example, when determining that the water level is enough to cover the pulverizing transfer part 20 in the treatment chamber 10, the controller 240 may close the water supply valve 40 to stop the supply of water.

According to an example, in operation 630, the controller 240 may generate an internal water flow by operating the pulverizing transfer part (e.g., the pulverizing transfer part 20 in FIG. 1). The controller 240 may rotate the pulverizing transfer part 20 by driving the first motor (e.g., the first motor 250 of FIG. 2). Due to the water flow formed by the rotation of the pulverizing transfer part 20, food waste adhered to the inner wall of the treatment chamber 10 or the surface of the pulverizing transfer part 20 may be washed away.

The controller 240 may generate an internal water flow by rotating the first motor 250 in the first direction R1 or the second direction R2. The controller 240 may alternately rotate the first motor 250 in the first direction R1 and the second direction R2 to generate a water flow. For example, the controller 240 may repeatedly perform the control of rotating the first motor 250 in the first direction R1 for a first time and then rotating the first motor 250 in the second direction R2 for the first time. For example, the controller 240 may repeatedly perform the operation of rotating the first motor 250 in the first direction R1 for the first time, stopping the rotation of the first motor 250 for a second time, and then rotating the first motor 250 in the second direction R2 for the first time. Here, the first time or the second time may be a value stored in the memory 242, and the second time may be shorter than the first time.

According to an example, in operation 640, the controller 240 may perform the control to open the drain valve 50 and rotate the pulverizing transfer part 20 in the first direction R1. After operation 630 is completed, the controller 240 may open the drain valve 50 to drain water. In the process of draining water, the controller 240 may rotate the pulverizing transfer part 20 in the first direction R1 to transfer food waste remaining in water by washing in operation 630. The controller 240 may adjust the degree of opening of the drain valve 50 to adjust the rate at which water is drained.

The terms as used herein are provided merely to describe some embodiments thereof, but are not intended to limit the disclosure. As used herein, the singular forms “a,” “an,” and “the” are intended to include the plural forms as well, unless the context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, the term ‘and/or’ should be understood as encompassing any and all possible combinations by one or more of the enumerated items. As used herein, the terms “include,” “have,” and “comprise” are used merely to designate the presence of the feature, component, part, or a combination thereof described herein, but use of the term does not exclude the likelihood of presence or adding one or more other features, components, parts, or combinations thereof. As used herein, the terms “first” and “second” may modify various components regardless of importance and/or order and are used to distinguish a component from another without limiting the components.

As used herein, the terms “configured to” may be interchangeably used with the terms “suitable for,” “having the capacity to,” “designed to,” “adapted to,” “made to,” or “capable of” depending on circumstances. The term “configured to” does not essentially mean “specifically designed in hardware to.” Rather, the term “configured to” may mean that a device can perform an operation together with another device or parts. For example, a ‘device configured (or set) to perform A, B, and C’ may be a dedicated device to perform the corresponding operation or may mean a general-purpose device capable of various operations including the corresponding operation.

Meanwhile, the terms “upper side”, “lower side”, and “front and rear directions” used in the disclosure are defined with respect to the drawings, and the shape and position of each component are not limited by these terms.

In the disclosure, the above-described description has been made mainly of specific embodiments, but the disclosure is not limited to such specific embodiments, but should rather be appreciated as covering all various modifications, equivalents, and/or substitutes of various embodiments.

What is claimed is:

1. A food waste disposal device comprising:
a treatment chamber comprising an inlet, a drain part connected to a sink drainpipe, and a byproduct outlet configured to have byproducts discharged therethrough;
a pulverizing transfer part rotatably disposed within the treatment chamber, and configured to grind and transfer an object introduced through the inlet;
a storage part configured to store the byproducts discharged through the byproduct outlet;
a drain valve configured to adjust an opening degree of the drain part; and
a controller configured to control operations of the pulverizing transfer part and the drain valve to perform:
in a first mode, an operation of opening the drain valve and rotating the pulverizing transfer part in a first direction to grind and transfer the object introduced through the inlet; and
in a second mode, an internal cleaning operation of operating the pulverizing transfer part in a state in which the drain valve is closed and water is received in the treatment chamber.

2. The food waste disposal device of claim 1, wherein the treatment chamber comprises a water inlet portion configured to allow externally supplied water to pass therethrough, and
wherein the food waste disposal device further comprises a water supply valve configured to control an opening and a closing of the water inlet portion.

3. The food waste disposal device of claim 2, wherein the controller is further configured to perform, in the second mode, an operation of opening the water supply valve to supply water into the treatment chamber.

4. The food waste disposal device of claim 1, further comprising an odor sensor configured to detect an odor signal indicating a concentration of malodorous components in the treatment chamber,
wherein the controller is further configured to perform the second mode in a state in which the concentration of malodorous components in the treatment chamber is equal to or greater than a preset level.

5. The food waste disposal device of claim 1, wherein the controller is further configured to perform, in the second mode, an operation of at least partially opening the drain valve and rotating the pulverizing transfer part in the first direction after performing the internal cleaning operation.

6. The food waste disposal device of claim 1, wherein the controller is further configured to perform, in the second mode, an operation of alternately rotating the pulverizing transfer part in the first direction and a second direction opposite to the first direction during the internal cleaning operation.

7. The food waste disposal device of claim 1, further comprising a strainer on the drain part and comprising a plurality of holes with a predetermined size.

8. The food waste disposal device of claim 1, further comprising a gasket configured to close the byproduct outlet, and to open the byproduct outlet in a state in which a pressure is applied that is equal to or greater than a predetermined level.

9. The food waste disposal device of claim 1, further comprising:
a blower fan configured to dry the byproducts stored in the storage part; and
a moisture exhaust pipe comprising a first end connected to an upper side of the storage part and a second end connected to the sink drainpipe.

10. A food waste disposal device comprising:
a treatment chamber comprising a first chamber having an open area, a second chamber coupled to the open area and having a cross-sectional area that decreases away from the first chamber, and a byproduct outlet at an end portion of the second chamber away from the first chamber; and
a pulverizing transfer part inside both the first chamber and the second chamber in the treatment chamber, and comprising a rotating body, a first blade extending from an outer circumferential surface of a portion of the rotating body in the first chamber, and a second blade extending from an outer circumferential surface of a portion of the rotating body positioned in the second chamber and having a shorter length than the first blade.

11. The food waste disposal device of claim 10, wherein the first blade is configured to rotate the rotating body to grind an object introduced into the treatment chamber, and
wherein the second blade is configured to compress and transfer the object ground by the rotating body to the byproduct outlet.

12. The food waste disposal device of claim 10, wherein the rotating body comprises a first body in the first chamber and a second body in the second chamber, and
wherein a thickness of the second body is larger than a thickness of the first body.

13. The food waste disposal device of claim 10, further comprising a gasket configured to close the byproduct outlet, and to open the byproduct outlet in a state in which a pressure is applied that is equal to or greater than a predetermined level.

14. The food waste disposal device of claim 10, further comprising:
a storage part configured to store byproducts discharged from the byproduct outlet;
a blower fan configured to dry byproducts stored in the storage part; and
a moisture exhaust pipe comprising a first end connected to an upper side of the storage part and configured to discharge moisture inside the storage part.

15. The food waste disposal device of claim 10, wherein the second chamber is conical or frustoconical in shape.
